(19) United States
(12) Patent Application Publication
(10) Pub. No.: US 2024/0108625 A1
(43) Pub. Date: Apr. 4, 2024

(54) CDK4/6 INHIBITORS AS A TREATMENT FOR CLONAL HEMATOPOIESIS

(71) Applicant: Washington University, St. Louis, MO (US)

(72) Inventor: Kelly Bolton, St. Louis, MO (US)

(73) Assignee: Washington University, St. Louis, MO (US)

(21) Appl. No.: 18/480,409

(22) Filed: Oct. 3, 2023

Related U.S. Application Data
(60) Provisional application No. 63/413,184, filed on Oct. 4, 2022.

(51) Int. Cl. A61K 31/519; A61P 7/00; A61P 35/00

(52) U.S. Cl. CPC A61K 31/519; A61P 7/00; A61P 35/00

(57) ABSTRACT

The present disclosure provides for compositions and methods of use of CDK4/6 inhibitors to prevent therapy-related clonal hematopoiesis and associated myeloid neoplasms.

### CROSS-REFERENCE TO RELATED APPLICATIONS

This application claims the benefit of priority to U.S. Provisional Application No. 63/413,184 filed on Oct. 4, 2022, the content of which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to compositions and methods of use for CDK4/6 inhibitors in the treatment of clonal hematopoiesis.

### BACKGROUND OF THE INVENTION

Myelodysplastic syndrome (MDS) and acute myeloid leukemia (AML) are fatal diseases that are highly resistant to therapy. The most established risk factors for MDS are advancing age and prior exposure to oncologic therapy given for a previous solid tumor. Therapy-related MN (tMN) accounts for 15-20% of MDS cases and is almost universally fatal. Clonal hematopoiesis (CH), whereby a hematopoietic stem or progenitor cell (HSPC) acquires a mutation that confers a selective advantage is common with normal human aging. However, in a small subset of individuals, CH clones will expand and acquire additional mutations, eventually leading to AML/MDS. Despite advances in the ability to identify individuals with CH and a high risk of MDS development based on genomic and clinical features, there are no established therapeutic strategies to target CH clones to prevent transformation.

Clonal hematopoiesis (CH) is common in middle-aged and elderly populations and confers a risk of hematological malignancy and death due to cardiovascular disease. Prior therapy with cytotoxic chemotherapy or radiation increases the risk of CH, especially that associated with TP53 or PPM1D mutations. CH can complicate the interpretation of cell-free or circulating tumor DNA assays since most blood DNA is derived from hematopoietic cells. The specific determinants of clonal progression are unclear, but the gene carrying the mutation, the size of the mutant clone, and the presence of multiple mutations appear to increase the risk of evolution to myeloid leukemia. While CH is not yet modifiable, specific mutations such as TET2 or IDH1/IDH2 confer vulnerabilities to established drugs or developmental compounds, and investigators are developing clinical trials to try to exploit these vulnerabilities.

### SUMMARY OF THE INVENTION

Among the various aspects of the present disclosure is the provision of compositions and methods of use for CDK4/6 inhibitors to prevent therapy-related clonal hematopoiesis.

Briefly, therefore, the present disclosure is directed to methods of use of CDK4/6 inhibitors to prevent clonal hematopoiesis and associated disorders.

A method of treatment to prevent or mitigate clonal hematopoiesis is disclosed that includes administering a therapeutic amount of a CDK4/6 inhibitor to a patient in need. In some aspects, the CDK4/6 inhibitor is selected from trilaciclib, palbociclib, ribociclib, and any combination thereof. In some aspects, the CDK4/6 inhibitor is trilaciclib. In some aspects, the clonal hematopoiesis is associated with the administration of chemotherapy to the patient. In some aspects, the chemotherapy is selected from carboplatin, etoposide, gemcitabine, FOLFOXIRI/bevacizumab, and any combination thereof. In some aspects, the method further includes administering a therapeutic amount of a CDK4/6 inhibitor to the patient concurrently with the chemotherapy. In some aspects, preventing or mitigating clonal hematopoiesis prevents or mitigates myeloid neoplasms. In some aspects, preventing or mitigating the clonal hematopoiesis prevents or mitigates a hematological malignancy or cardiovascular disease associated with clonal hematopoiesis.

In another aspect, a method of treatment to prevent or mitigate a hematologic cancer associated with clonal hematopoiesis is disclosed that includes administering a therapeutic amount of a CDK4/6 inhibitor to a patient in need. In some aspects, the hematologic cancer is selected from myelodysplastic syndrome (MDS) and acute myeloid leukemia (AML). In some aspects, the CDK4/6 inhibitor is selected from trilaciclib, palbociclib, ribociclib, and any combination thereof. In some aspects, the CDK4/6 inhibitor is trilaciclib. In some aspects, administering the therapeutic amount of the CDK4/6 inhibitor prevents or mitigates clonal hematopoiesis. In some aspects, the clonal hematopoiesis is associated with the administration of chemotherapy to the patient. In some aspects, the chemotherapy is selected from carboplatin, etoposide, gemcitabine, FOLFOXIRI/bevacizumab, and any combination thereof. In some aspects, the method further includes administering the therapeutic amount of the CDK4/6 inhibitor to the patient concurrently with the chemotherapy. In some aspects, administering the therapeutic amount of a CDK4/6 inhibitor further prevents or mitigates myeloid neoplasms associated with clonal hematopoiesis. In some aspects, administering the therapeutic amount of a CDK4/6 inhibitor further prevents or mitigates a cardiovascular disease associated with clonal hematopoiesis.

Other objects and features will be in part apparent and in part pointed out hereinafter.

### DESCRIPTION OF THE DRAWINGS

The following drawings illustrate various aspects of the disclosure.

FIG. 1 is a schematic illustration showing an experimental design for a clinical trial to assess the efficacy of carboplatin with either trilaciclib or a placebo for the treatment of small cell lung cancer as described in the examples below.

FIG. 2 is a schematic illustration summarizing an experimental design for Artifact Filtering and Consensus Clonal Hematopoiesis Calling Pipeline (ArCCH) used to analyze the sequencing data obtained as described in FIG. 1.

FIG. 3A is a histogram summarizing the distribution of samples having 1 or more CH mutations within the two small cell lung cancer patient populations illustrated in FIG. 1.

FIG. 3B is a histogram summarizing the distribution of mutations within selected genes of the two small cell lung cancer patient populations illustrated in FIG. 1.

FIG. 4 is a graph summarizing the exponential growth slopes of mutations of various DDR genes of clonal hematopoiesis.

FIG. 5 is a graph summarizing the exponential growth slopes of mutations of various driver genes of clonal hematopoiesis.

FIG. 6 is a schematic illustration summarizing an experimental design to assess the impact of a variety of CDK34/6 inhibitors in TP53 and TET2 mutant CH murine models.

FIG. 7 is a schematic illustration summarizing an experimental design for a competitive transplant to assess CH fitness under CDK4/6 inhibition. showing the Trp53-R^{172H+/-} experiment with Trilaciclib. An identical design will be used for Tet2 KO and alternative CDK4/6 inhibitors.

Those of skill in the art will understand that the drawings, described below, are for illustrative purposes only. The drawings are not intended to limit the scope of the present teachings in any way.

### DETAILED DESCRIPTION OF THE INVENTION

The present disclosure is based, at least in part, on the discovery that Trilaciclib inhibited carboplatin-related clonal hematopoiesis. As shown herein, Trilaciclib treatment protects against both DDR and DTA clonal hematopoiesis related to carboplatin chemotherapy.

Clonal hematopoiesis (CH) is a process whereby hematopoietic stem and progenitor cells acquire mutations in MDS driver genes that lead to selective advantage and clonal expansion. CH is a risk factor for MDS and longitudinal studies in humans suggest that CH is the precursor of MDS. Prior studies from our group and others have identified that environmental stressors, such as exposure to cytotoxic chemotherapy or radiation, confer an even higher selection advantage on CH clones and risk of transformation to MDS. While individuals with CH who are at high-risk of progression to MDS can be identified based on clinical and CH mutational characteristics, at present there are no established interventional strategies to eradicate CH and mitigate the risk of transformation to MDS. Due to the difficulty in treating MDS, early intervention through prevention may ultimately be the highest yield strategy to eradicate this lethal disease.

Clonal hematopoiesis (CH), whereby a hematopoietic stem or progenitor cell (HSPC) acquires a mutation that confers a selective advantage, is common with human aging. However, in a small subset of individuals, CH clones will expand and acquire additional mutations, eventually leading to hematologic cancers, particularly MDS. CH mutations confer a fitness advantage through multiple pathways. Loss-of-function mutations in the epigenetic regulators DNMT3A, TET2, and ASXL1 (so-called “DTA” mutations) are the most common

CH drivers and result in increased HSPC self-renewal, pro-myeloid differentiation, and an exacerbated inflammatory phenotype7-9. Mutations in DNA damage response (DDR) pathway members, including TP53 and PPM1D, confer an advantage through suppression of apoptosis in response to DNA damage. Thus, DDR CH clones show a fitness advantage in the setting of environmental stressors such as cytotoxic therapy. We and others have previously shown that the expansion of TP53-mutant CH under the selective pressure of cytotoxic therapy is the major driver of tMN.

CDK4/6 inhibitors act by maintaining the cell in the G1 phase and limiting DNA replication. The transient arrest of the cell cycle can be beneficial for hematopoietic stem and progenitor cells exposed to cytotoxic chemotherapy because it avoids their proliferation in a toxic environment and prevents bone marrow exhaustion. As such, Trilaciclib is FDA-approved to prevent chemotherapy-induced myelosuppression in patients with small cell lung cancer.

Trilaciclib is an intravenous cyclin-dependent kinase CDK4/6 inhibitor that transiently arrests CDK4/6-dependent HSPCs in the G1 phase of the cell cycle. When given during chemotherapy exposure, trilaciclib protects HSPCs from chemotherapy-induced damage. Trilaciclib is currently FDA-approved as an adjunctive therapy in small cell lung cancer (SCLC) patients receiving a platinum/etoposide or topotecan-containing regimen to decrease the incidence of chemotherapy-induced myelosuppression.

While diverse pathways drive the competitive advantage of specific CH mutations, a common mechanism in CH pathogenesis is cell-cycle regulation of HSPCs which is known to be tightly controlled by CDK4/6. Consequently, it is thought that CDK4/6 inhibition would likely inhibit chemotherapy-induced expansion of CH mutations.

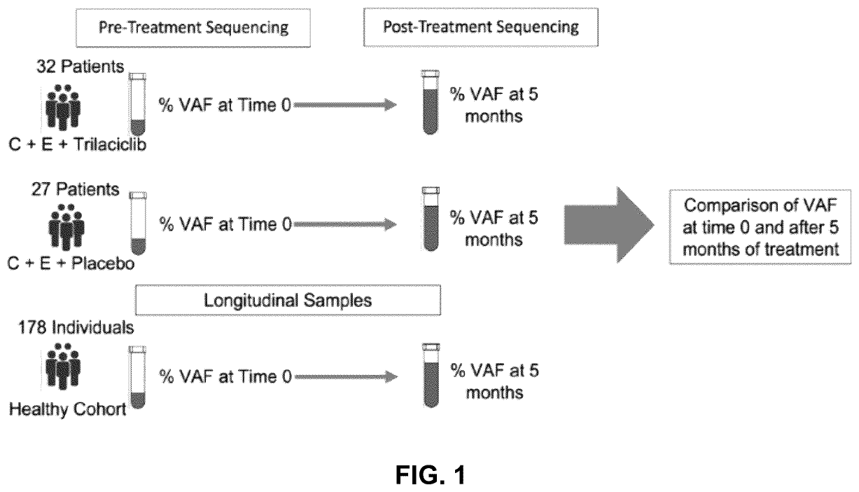

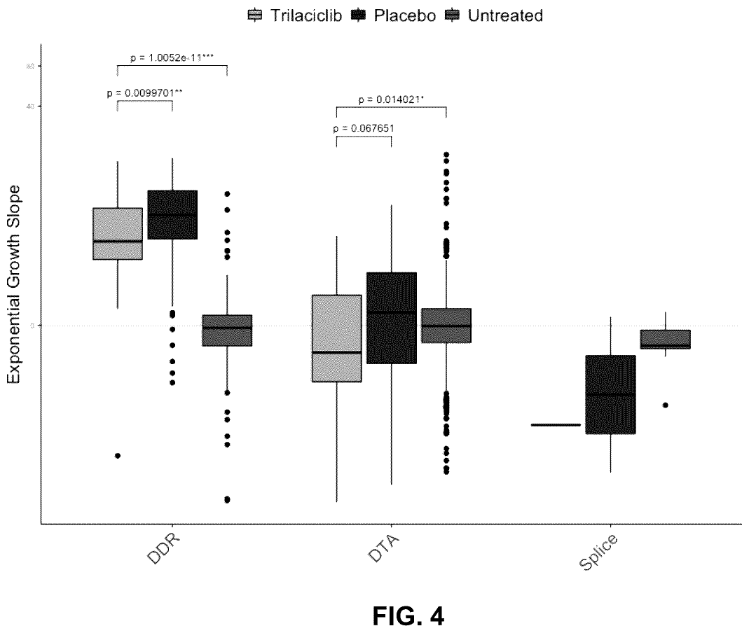

As demonstrated in the examples, pharmacologic inhibition of CDK4/6 shows promise in mitigating CH expansion. Genomic analyses of blood samples from patients with small cell lung cancer (SCLC) on a clinical trial and randomized to receive carboplatin/etoposide combined with the CDK4/6 inhibitor trilaciclib or placebo (see FIG. 1) revealed that lung cancer patients with high-risk CH mutations in the DNA Damage Response pathway (TP53, CHEK2, PPM1D) who received CDK4/6 inhibition showed a lower expansion with chemotherapy (FIG. 5). It was also observed that CDK4/6 inhibition suppressed growth of CH clones bearing mutations in the epigenetic modifiers (DNMT3A, TET2, ASXL1). The results of these experiments suggested that CDK4/6 inhibition may show promise as a therapeutic agent to prevent the progression of CH to MDS. Based on these data, it was thought that CDK4/6 inhibition extended beyond the protection of HSPCs during chemotherapy to prevent CH expansion and transformation to overt MDS.

One aspect of the present disclosure provides compositions of CDK4/6 inhibitors. In some aspects, a CDK4/6 inhibitor prevents clonal hematopoiesis, which can be associated with a therapy including but not limited to chemotherapy.

Another aspect of the present disclosure is a method of treatment that includes the administration of a therapeutic amount of a CDK4/6 inhibitor to prevent or mitigate clonal hematopoiesis, which can be associated with a therapy including but not limited to chemotherapy. In some aspects, preventing or mitigating clonal hematopoiesis prevents therapy-related myeloid neoplasms. In some aspects, the CDK4/6 inhibitor can be administered before, during, or after another therapy, which can be a chemotherapy such as carboplatin. In some embodiments, the CDK4/6 inhibitor can be trilaciclib, palbociclib, ribociclib, and any other suitable CDK4/6 inhibitor without limitation.

In some aspects, treatment response monitoring can be performed. In some embodiments, treatment response monitoring is performed with DNA sequencing. In some embodiments, ArcherDX Custom 9 Gene UMI-based Panels are used. In some embodiments, the DMNT3A, TET2, ASXL1, PPM1D, TP53, CHEK2, SRSF2, SF3B1, and JAK2 genes can be characterized with DNA sequencing.

In some aspects, ArCCH: Artifact Filtering and Consensus Clonal Hematopoiesis Calling Pipeline (ArCCH) can be performed which can feature 3 variant callers: Mutect2, Vardict, and Lofreq2. Additionally, Pindel can be run as a validation check for possible complex indel support. Variants from these callers can be combined together in a consensus whereby it is required a variant to be passed by one or more callers. Each variant can then be filtered through various false positive filtering parameters as well as statistically tested against our Panel of Normal. Variants passing these filters can then be annotated using VEP as well as our in-house custom CH putative driver annotation script in which all of the resulting information would be merged together and consolidated for manual review.

In some aspects, a treatment is performed on a subject. In some aspects, these patients have at least one clonal hematopoiesis mutation. In some embodiments, the subject has an enrichment of DDR or DTA genes. In some embodiments, the DDR genes can be PPM1D, TP53, or CHEK2. In some aspects, the CDK4/6 inhibitor, which can include but is not limited to trilaciclib, can protect against DDR or DTA clonal hematopoiesis and decrease overall clonal hematopoiesis growth in short-term follow-up. In some aspects, the reduction in growth rate can be similar between DDR genes and other driver genes. However, Trilaciclib can have a significant effect on decreasing the overall growth rate of clonal hematopoiesis mutations as compared to untreated individuals. In some aspects, this can be a short-term or long-term effect of the drug. In some aspects, there can be a decreasing effect of Trilaciclib that is similar between all genes with a strong negative clonal hematopoiesis selection in driver genes. Trilaciclib can have a dampening effect on CH expansion typically caused by carboplatin on the DDR genes, with a clear advantageous reduction in other driver genes. In summary, Trilaciclib shows promise in mitigating the expansion of clonal hematopoiesis, which is the first therapeutic approach to prevent clonal hematopoiesis from progressing to myeloid neoplasm.

CDK4/6 Modulation Agents

As described herein, CDK4/6 expression has been implicated in various diseases, disorders, and conditions. As such, modulation of CDK4/6 (e.g., modulation of DDR) can be used for the treatment of such conditions. A CDK4/6 modulation agent can modulate CDK4/6 response or induce or inhibit CDK4/6. CDK4/6 modulation can comprise modulating the expression of CDK4/6 on cells, modulating the quantity of cells that express CDK4/6, or modulating the quality of the CDK4/6 cells.

CDK4/6 modulation agents can be any composition or method that can modulate CDK4/6 expression on cells (e.g., Trilaciclib). For example, a CDK4/6 modulation agent can be an activator, an inhibitor, an agonist, or an antagonist. As another example, the CDK4/6 modulation can be the result of gene editing.

A CDK4/6 modulation agent can be a CDK4/6 antibody (e.g., a monoclonal antibody to CDK4/6).

A CDK4/6 modulating agent can be an agent that induces or inhibits progenitor cell differentiation into CDK4/6 expressing cells (e.g., hematopoietic clones). For example, Trilaciclib can be used to block CDK4/6.

CDK4/6 Signal Reduction, Elimination, or Inhibition by Small Molecule Inhibitors, shRNA, siRNA, or ASOs

As described herein, a CDK4/6 modulation agent can be used for use in cancer chemotherapy. A CDK4/6 modulation agent can be used to reduce/eliminate or enhance/increase CDK4/6 signals. For example, a CDK4/6 modulation agent can be a small molecule inhibitor of CDK4/6. As another example, a CDK4/6 modulation agent can be a short hairpin RNA (shRNA). As another example, a CDK4/6 modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

CDK4/6 Inhibiting Agent

One aspect of the present disclosure provides for targeting of CDK4/6, its receptor, or its downstream signaling. The present disclosure provides methods of treating or preventing therapy-related clonal hematopoiesis expansion to prevent myeloid neoplasms based on the discovery that Trilaciclib shows promise in mitigating the expansion of clonal hematopoiesis.

As described herein, inhibitors of CDK4/6 (e.g., antibodies, fusion proteins, small molecules) can reduce or prevent clonal hematopoiesis. A

CDK4/6 inhibiting agent can be any agent that can inhibit CDK4/6, downregulate CDK4/6, or knockdown CDK4/6.

As an example, a CDK4/6 inhibiting agent can inhibit CDK4/6 signaling.

For example, the CDK4/6 inhibiting agent can be an anti-CDK4/6 antibody. As an example, the anti-CDK4/6 antibody can be an anti-CDK4/6 antibody, an anti-DDR antibody, or an anti-DTA antibody with activity against any combination of CDK4/6, DDR, or DTA. Furthermore, the anti-CDK4/6 antibody can be a murine antibody, a humanized murine antibody, or a human antibody.

As another example, the CDK4/6 inhibiting agent can be an anti-CDK4/6 antibody, wherein the anti-CDK4/6 antibody prevents binding of CDK4/6 to its receptor or prevents activation of CDK4/6 and downstream signaling.

As another example, the CDK4/6 inhibiting agent can be a fusion protein. For example, the fusion protein can be a decoy receptor for CDK4/6. Furthermore, the fusion protein can comprise a mouse or human Fc antibody domain fused to the ectodomain of CDK4/6.

As another example, a CDK4/6 inhibiting agent can be Trilaciclib, which has been shown to be a potent and specific inhibitor of CDK4/6 signaling.

As another example, a CDK4/6 inhibiting agent can be an inhibitory protein that antagonizes CDK4/6. For example, the CDK4/6 inhibiting agent can be a viral protein, which has been shown to antagonize CDK4/6.

As another example, a CDK4/6 inhibiting agent can be a short hairpin RNA (shRNA) or a short interfering RNA (siRNA) targeting CDK4/6 or DDR.

As another example, a CDK4/6 inhibiting agent can be an sgRNA targeting CDK4/6 or DDR.

Any suitable CDK4/6 modulation agent used in the treatment methods described herein including, but not limited to, trilaciclib, palbociclib, ribociclib, any combination thereof, and any other suitable CDK4/6 modulation agent without limitation.

Methods for preparing a CDK4/6 inhibiting agent (e.g., an agent capable of inhibiting CDK4/6 signaling) can comprise the construction of a protein/Ab scaffold containing the natural CDK4/6 receptor as a CDK4/6 neutralizing agent; developing inhibitors of the CDK4/6 target “down-stream”; or developing inhibitors of the CDK4/6 production “up-stream”.

Inhibiting CDK4/6 can be performed by genetically modifying CDK4/6 in a subject or genetically modifying a subject to reduce or prevent expression of the CDK4/6 gene, such as through the use of CRISPR-Cas9 or analogous technologies, wherein, such modification reduces or prevents clonal hematopoiesis.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit the translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities, and retaining a required activity of the expressed protein are within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid that is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in vitro using the specific codon usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tm=81.5° C.+16.6(log₁₀[N⁺)+0.41(fraction G/C content)−0.63(% formamide)−(600/I). Furthermore, the Tm of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Genome Editing

As described herein, CDK4/6 signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of CDK4/6 by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double-strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for the prevention of therapy-related clonal hematopoiesis to target cells by the removal of CDK4/6 signals.

For example, the methods as described herein can comprise a method for altering a target polynucleotide sequence in a cell comprising contacting the polynucleotide sequence with a clustered regularly interspaced short palindromic repeats-associated (Cas) protein.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption-delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

Therapeutic Methods

Also provided is a process of treating, preventing, or reversing clonal hematopoiesis in a subject in need of administration of a therapeutically effective amount of a CDK4/6 inhibiting agent, so as to prevent myeloid neoplasms, cardiovascular disorders, or any other disorder associated with clonal hematopoiesis without limitation.

Methods described herein are generally performed on a subject in need thereof. A subject in need of the therapeutic methods described herein can be a subject having, diagnosed with, suspected of having, or at risk for developing myeloid neoplasms. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a CDK4/6 inhibiting agent is, for example, an amount that would cause the desired therapeutic effect in a subject while minimizing undesired side effects. In various embodiments, an effective amount of a CDK4/6 inhibiting agent described herein can substantially inhibit clonal hematopoiesis, slow the progress of clonal hematopoiesis, or limit the development of clonal hematopoiesis.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a CDK4/6 inhibiting agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to prevent clonal hematopoiesis.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

The toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Shargel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treatment can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a CDK4/6 inhibiting agent can occur as a single event or over a time course of treatment. For example, a CDK4/6 inhibiting agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for therapy-related myeloid neoplasms.

A CDK4/6 inhibiting agent can be administered simultaneously or sequentially with another agent, such as an antibiotic, an anti-inflammatory, or another agent. For example, a CDK4/6 inhibiting agent can be administered simultaneously with another agent, such as an antibiotic or an anti-inflammatory. Simultaneous administration can occur through the administration of separate compositions, each containing one or more of a CDK4/6 inhibiting agent, an antibiotic, an anti-inflammatory, a chemotherapy, or another agent. Simultaneous administration can occur through the administration of one composition containing two or more of a CDK4/6 inhibiting agent, an antibiotic, an anti-inflammatory, a chemotherapy, or another agent. A CDK4/6 inhibitor can be administered sequentially with an antibiotic, an anti-inflammatory, a chemotherapy, or another agent. For example, a CDK4/6 inhibiting agent can be administered before or after the administration of an antibiotic, an anti-inflammatory, a chemotherapy, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., a molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., a molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8A to about 15A.

Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to a CDK4/6 inhibitor, a chemotherapy composition, and a stabilizing agent. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water and sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice.

However, those of skill in the art should, in light of the present disclosure, appreciate that many changes can be made in the specific embodiments that are disclosed and still obtain a like or similar result without departing from the spirit and scope of the present disclosure.

### Example 1: Genomic Analysis of Small Cell Lung Cancer Patients Receiving Chemotherapy+CDK4/6 Inhibitor

To evaluate the effect of trilaciclib on the expansion of CH clones in patients receiving chemotherapy, the following experiments were conducted. Blood samples obtained from a randomized, placebo-controlled trial of trilaciclib in SCLC patients receiving fist-line carboplatin and etoposide chemotherapy were sequenced pre- and post-treatment.

Genomic analyses of the blood of patients with small cell lung cancer (SCLC) who were on a clinical trial and randomized to receive carboplatin/etoposide combined with the CDK4/6 inhibitor trilaciclib or placebo were performed. CDK4/6 inhibitors act by maintaining the cell in the G1 phase and limiting DNA replication. The transient arrest of the cell cycle can be beneficial for hematopoietic stem and progenitor cells exposed to cytotoxic chemotherapy because it avoids their proliferation in a toxic environment and prevents bone marrow exhaustion. Trilaciclib is FDA-approved to prevent chemotherapy-induced myelosuppression in patients with small cell lung cancer.

Blood samples were obtained from a clinical trial of SCLC patients receiving first-line carboplatin/etoposide combination chemotherapy randomized to concurrently receive the CDK4/6 inhibitor trilaciclib or placebo (FIG. 1). We performed targeted mutational analysis of genomic DNA from peripheral blood before and after five cycles of carboplatin/etoposide (˜5 months) in 59 individuals, 32 of whom received concurrent trilaciclib with each cycle of chemotherapy (FIG. 1, top row) and 27 who received placebo (FIG. 1, middle row). As a comparator group, sequential samples were obtained from 178 individuals from another longitudinal study (FIG. 1, bottom row) selected as cancer-free and matched in age and length of follow-up to the participants in the SCLC clinical trial. All samples were analyzed as described below to measure how the number of HSPCs bearing CH mutations (reflected by the variant allele frequency [VAF]) changed over time in individuals randomized to receive trilaciclib compared to placebo and to untreated healthy adults.

To accurately characterize clonal evolution over time, a CH assay was developed that showed excellent reproducibility and precision in regards to VAF and was able to detect very low VAF variants. A custom-targeted CH panel was developed using VariantPlex (ArcherDX), an amplicon sequencing method that leverages bi-directional primers and molecular barcodes (MBCs) to enable reliable detection of extremely low VAF variants. The CH panel included exonic regions of the nine CH genes most commonly mutated in solid tumor patients (DNMT3A, TET2, ASXL1, PPM1D, TP53, CHEK2, JAK2, SRSF2, and SF3B1).

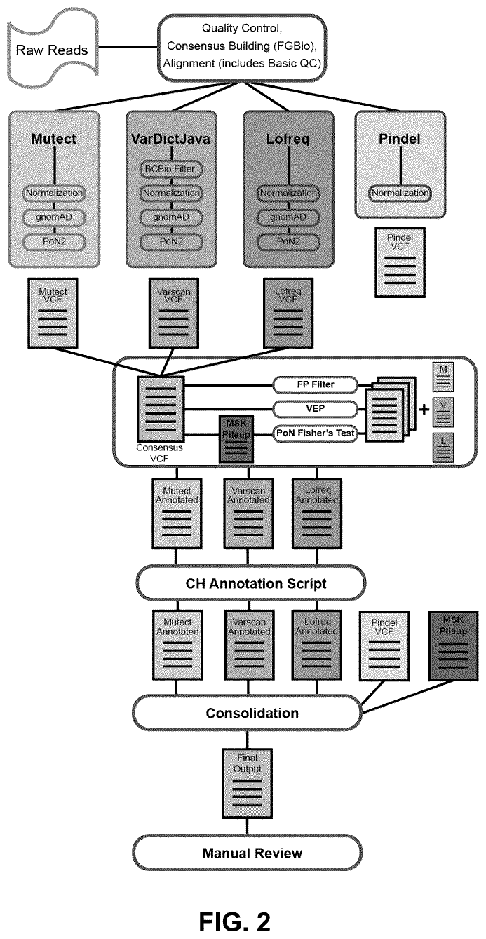

The workflow for the CH assay is summarized in FIG. 2. In brief, following DNA fragmentation, ligation with a universal MBC adapter was performed, tagging each DNA molecule with a unique molecular index (UMI) and allowing for unidirectional amplification of the sample using gene-specific primers. The resulting libraries were sequenced using a NovaSeq 6000 instrument. The average unique coverage per sample was 20,746×. Using tumor-normal dilution series from six AML patients, we developed a custom CH variant calling pipeline integrating three variant callers (Mutect2, Vardict, LoFreq) with post-calling filtering to remove germ line variants and artifacts. A critical feature of this pipeline is the empiric estimation of variant-specific error rates using a technical panel of normals consisting of 30 blood samples from children18. Applying this custom pipeline to a validation cohort consisting of 31 blood samples from healthy individuals sequenced using VariantPlex with variants validated using an orthogonal approach, we saw excellent sensitivity (0.99 [>1% VAF], 0.92 [0.2-1%] and positive predictive value (1.00 [>1% VAF], 0.98 [0.2-1%]). In technical replicates, we showed excellent precision for VAF, including low VAF variants between 1% and 0.2% VAF (R2=99%). Thus, in summary, our custom CH panel with the associated bioinformatics pipeline shows excellent sensitivity, specificity, and precision for the detection of CH as low as 0.2% VAF, making it well suited for studying the impact of trilaciclib on CH evolution.

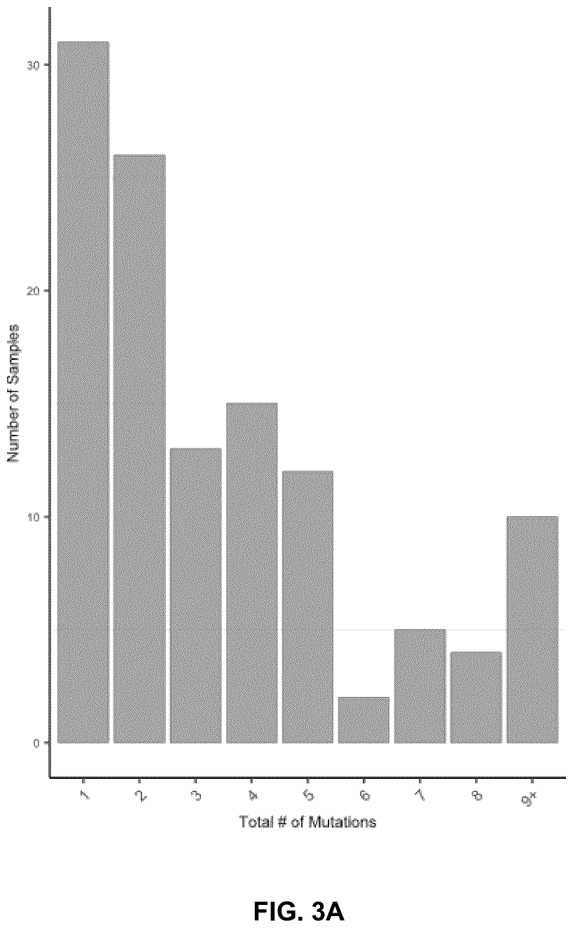

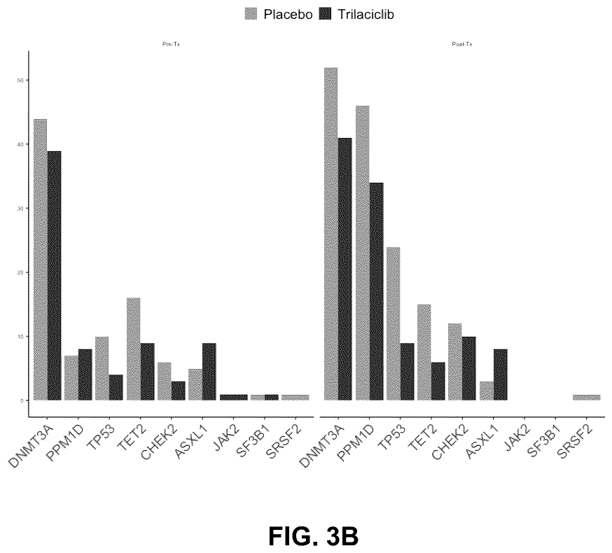

At the very high sequencing depth utilized, at least one CH mutation present at 0.2% VAF was observed in both the pre- and post-time points in 98% of cases among the 59 SCLC cases with a median age of 66 years at the first time point, as summarized in FIG. 3A. The distribution of mutations within genes was characterized by notable enrichments for DDR genes (PPM1D, TP53, CHEK2) relative to DTA genes. For each CH mutation, the change in VAF between the first and second time points was calculated. Individuals with CH mutations in the DDR pathway (TP53, CHEK2, PPM1D) who received CDK4/6 inhibition had reduced expansion following chemotherapy (FIG. 4) relative to the placebo group. Moreover, CDK4/6 inhibition suppressed the growth of CH clones bearing mutations in epigenetic modifiers DTA, as summarized in FIG. 5. Referring to FIG. 5, the largest impact of this growth suppression was observed for TET2. Thus, this preliminary work suggests that CDK4/6 inhibition may show promise as a therapeutic agent to prevent the progression of multiple genetic forms of CH to MN. Based on this promising preliminary data, we hypothesize that CDK4/6 inhibition extends beyond the protection of HSPCs during chemotherapy to prevent CH expansion and transformation to overt MDS. Here, we propose to 1) further characterize the potential of trilaciclib to prevent CH expansion in additional clinical trials and whether this reduction persists following completion of therapy, and 2) evaluate the potential of CDK4/6 inhibitors beyond trilaciclib to inhibit CH independent of chemotherapy using genetically engineered murine models of CH in TP53 and TET2.

The results of these experiments indicate that CDK4/6 inhibition using a therapeutic agent such as trilaciclib is a promising approach to prevent the progression of CH to MDS. Based on these data, it is thought that CDK4/6 inhibition extends beyond the protection of HSPCs during chemotherapy to the prevention of CH expansion and transformation to overt MDS.

### Example 2: Impact of CDK4/6 Inhibition on CH Expansion in Solid Tumor Patients Receiving Chemotherapy

To test the hypothesis that CDK4/6 inhibition will mitigate CH expansion in solid tumor patients receiving cytotoxic therapy and that this effect will persist following therapy completion, the following experiments will be conducted. The impact of CDK4/6 inhibition on CH will be assessed through the analysis of two additional randomized clinical trials of Trilaciclib in patients with breast cancer and colorectal cancer. Additionally, blood samples obtained 3-6 months following completion of CDK4/6 inhibitor therapy will be sequenced to determine whether the inhibitory effect of CDK4/6 inhibition extended beyond therapy completion. Without being limited to any particular hypothesis, CDK4/6 inhibition is thought to mitigate CH expansion in solid tumor patients receiving cytotoxic therapy and will persist following the completion of therapy.

Additional pre- and post-treatment samples drawn from two additional randomized clinical trials from individuals with breast and colon cancer will be analyzed. The analysis will be expanded to include samples collected several months following completion of therapy in these three randomized trials

Pre-post-treatment samples drawn from two additional randomized clinical trials of trilaciclib will be analyzed. The first trial will be the PRESERVE2 study, a placebo-controlled trial of trilaciclib vs. placebo administered prior to gemcitabine and carboplatin in patients with metastatic TNBC. The first trial will be the PRESERVE1 study, a randomized placebo-controlled trial of trilaciclib vs. placebo administered prior to FOLFOXIRI/bevacizumab in patients with metastatic colorectal cancer.

Within the PRESERVE2 study, Cl D1 samples (pre-treatment) and end-of-treatment samples (˜7 to 20 months on therapy) have been obtained for 42 individuals. Within the PRESERVE1 study, Cl D1 samples (pre-treatment) and end-of-treatment samples (˜9 months on therapy) have been obtained for 326 individuals. Within the SCLC trial and PRESERVE2, samples obtained 3-6 months following the completion of Trilaciclib and chemotherapy are available for 85 participants. In total, 463 samples drawn from the three randomized clinical trials of Trilaciclib will be sequenced. As a comparator group, samples from 350 age-matched healthy individuals with a comparable duration of follow-up will be sequenced.

All samples will be sequenced using the custom Archer VariantPlex assay described previously in Example 1. This panel includes exonic regions of the nine CH genes most commonly mutated in solid tumor patients (DNMT3A, TET2, ASXL1, PPM1D, TP53, CHEK2, JAK2, SRSF2, and SF3B1. Sample and library preparation, sequencing, and bioinformatic pipelines will be similar to the corresponding information provided in Example 1. Using this approach CH mutations as low as 0.2% VAF will be detected.

For each CH mutation, the growth rate for CH, defined as the change in VAF per month between the pre- and post-treatment samples, will be calculated. Generalized estimating equations adjusted by clinical trial will be used to test for differences in the CH growth rates between individuals randomized to trilaciclib compared to placebo and the untreated healthy cohort adjusting for age, gender, and race, and accounting for the potential correlation between the change in VAF of the CH mutations in the same person. Testing to determine whether the growth rate for CH mutations differs between individuals randomized to the trilaciclib and placebo arms and between the untreated, healthy individuals will be conducted. We will study the difference in the CH growth rate between groups separately for DDR and DTA CH. In secondary analyses, we will study the difference in CH growth rate for specific genes.

If the differences in the growth rate observed in the SCLC trial are consistent with the corresponding differences within the PRESERVE1 and 2 cohorts will result in greater than 90% power to detect a similar effect size with an alpha level of 0.05 in both the DDR (99% power) and DTA analyses (93% power).

The persistence of the stabilizing effect of Trilaciclib following the completion of therapy will also be evaluated. The growth rate of CH (separately by DTA and DDR mutations) will be compared during the post-treatment interval to the on-treatment interval through a paired t-test. It is thought that in the post-treatment follow-up interval, the Trilaciclib group will continue to show a similar or lower growth rate compared to placebo thus showing that the protective effects persist following completion of therapy. Based on a sample size of 95 individuals during treatment and post-treatment FU timepoints (40 placebo and 45 trilaciclib arm), 80% power will be achieved to detect a similar or lower growth rate for CH mutations in the Trilaciclib compared to placebo arms (effect size dz=0.26) with an alpha level of 0.05 using a one-sided paired t-test.

The results of these experiments will demonstrate that CDK4/6 inhibition will mitigate CH expansion in solid tumor patients receiving cytotoxic therapy and that this effect will persist following therapy completion

### Example 3: Impact of CDK4/6 Inhibition on CH Expansion in Murine Models Independent of Chemotherapy

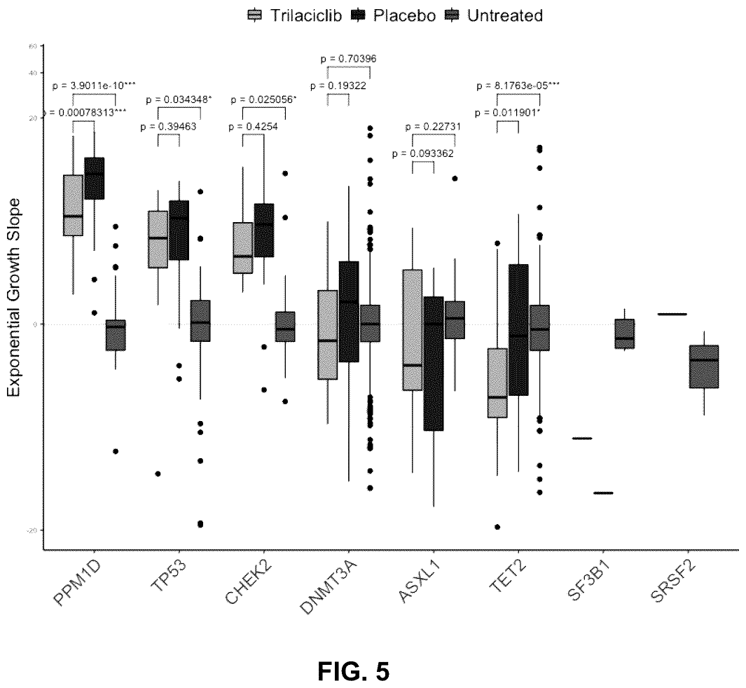

To determine whether CDK4/6 inhibition by administration of trilaciclib or other CDK4/6-inhibiting compounds attenuates the growth of CH clones independent of co-concurrent cytotoxic therapy, the following experiments will be conducted. Using genetically engineered murine models of TP53 and TET2 mutant CH we will test the impact of CDK4/6 inhibition in vivo in the presence or absence of concurrent chemotherapy, as summarized in FIG. 6. In addition, the impact of CDK4/6 inhibition on CH mitigation using additional CDK4/6 inhibitors including Palbociclib and Ribociclib will be similarly tested. Without being limited to any particular theory, it is thought that CDK4/6 inhibition using multiple agents will abrogate the competitive advantage of both TET2 KO and TP53^{R172H} HSPCs both in the presence and absence of concurrent chemotherapy.

Functional studies in genetically engineered murine models of TP53 and TET2 mutant CH will be used to perform studies of the impact of Trilaciclib, Palbociclib, and Ribociclib on the growth of CH mutant clones using competitive repopulation assays in vivo. The CDK4/6 inhibitors Palbociclib and Ribociclib were specifically chosen since they are the most commonly used CDK4/6 inhibitors in clinical practice with well-established dosing in mice.

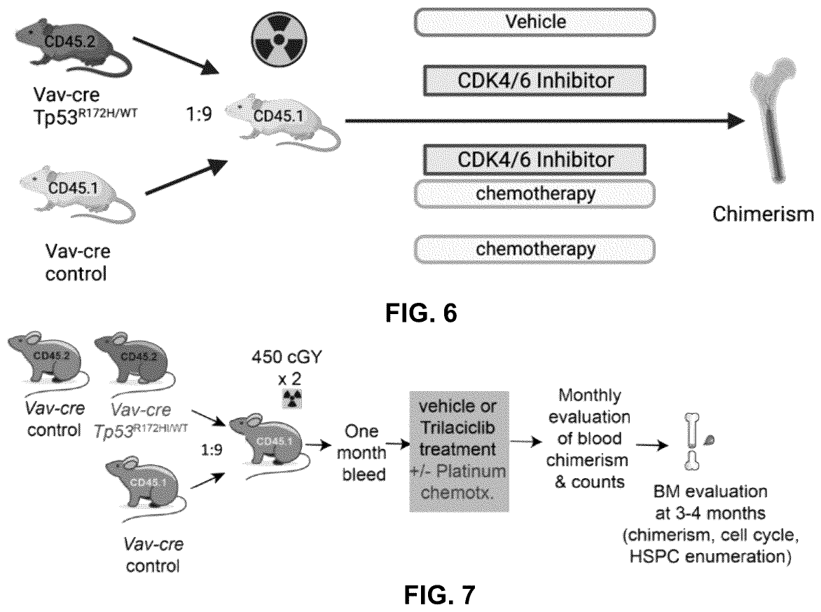

Competitive transplant of TP53^{R172H/WT} CD45.2 mice, WT CD45.1 competitor mice, and Tet2 KO CD45.2 mice/WT CD45.1 competitor mice will be performed, as illustrated in FIG. 6. The competitive advantage of TP53^{R172H} and Tet2 KO HSPCs will be assessed in cohorts of mice receiving vehicle, carboplatin+/−CDK4/6 inhibitor, and CDK4/6 inhibitor alone, as illustrated in FIG. 7. It is thought that CDK4/6 inhibition using multiple agents will abrogate the competitive advantage of both TET2 KO and TP53R172H HSPCs both in the presence and absence of concurrent chemotherapy.

FIG. 7 illustrates the experimental design for the trp53^{R172H} competitive transplant with Trilaciclib. A similar design will be used for the Tet2 KO model and the two other CDK4/6 inhibitors (Palbociclib and Ribociclib). Freshly dissected femurs and tibias will be isolated from Vav-cre, Vav-cre/trp53^{R172H/+}, or Vav-cre Tet2^{fl/fl} CD45.2+ mice and mixed in a 1:9 ratio with WT CD45.1⁺ BM and transplanted via tail-vein injection into 8-week-old lethally irradiated (900 cGy) CD45.1⁺ recipient mice. Four different exposure cohorts will be assessed: placebo alone, carboplatin+CDK4/6 inhibitor, CDK4/6 inhibitor alone, and carboplatin alone. Peripheral blood chimerism will be assessed every 4 weeks by flow cytometry to evaluate for changes in the ratios of CD45.1/CD45.2, blood counts, types of mature myeloid and lymphoid cells, megakaryocyte, and erythroid progenitors. At 4 months, the cohort will be sacrificed with bone marrow evaluation for chimerism, cell cycle, and HSPC enumeration. This experiment will help characterize whether CDK4/6 inhibition extends beyond the protection of HSPCs during chemotherapy to prevent CH expansion independently from CDK4/6 inhibition. The in vivo competitive transplantation assays described above will be done in biological triplicate to assess reproducibility with 10 mice per group in each arm of each experiment (with both male and female trp^{53R172H} and tet2 KO mice being used as donors). With 10 mice in each group, 80% power to detect a 20% difference in defined cell populations is achieved assuming an effect size (Op/6) of 2.0 between groups using a Wilcoxon rank-sum test at the 0.05 significance level.

The results of these experiments will demonstrate the potential of CDK4/6 inhibitors to prevent the progression of CH to MDS.

What is claimed is:

1. A method of treatment to prevent or mitigate clonal hematopoiesis, the method comprising administering a therapeutic amount of a CDK4/6 inhibitor to a patient in need.

2. The method of claim 1, wherein the CDK4/6 inhibitor is selected from trilaciclib, palbociclib, ribociclib, and any combination thereof.

3. The method of claim 2, wherein the CDK4/6 inhibitor is trilaciclib.

4. The method of claim 1, wherein the clonal hematopoiesis is associated with an administration of a chemotherapy to the patient.

5. The method of claim 4, wherein the chemotherapy is selected from carboplatin, etoposide, gemcitabine, FOLFOXIRI/bevacizumab, and any combination thereof.

6. The method of claim 4, further comprising administering a therapeutic amount of a CDK4/6 inhibitor to the patient concurrently with the chemotherapy.

7. The method of claim 1, wherein preventing or mitigating the clonal hematopoiesis prevents or mitigates myeloid neoplasms.

8. The method of claim 1, wherein preventing or mitigating the clonal hematopoiesis prevents or mitigates a hematological malignancy or cardiovascular disease associated with clonal hematopoiesis.

9. A method of treatment to prevent or mitigate a hematologic cancer associated with clonal hematopoiesis, the method comprising administering a therapeutic amount of a CDK4/6 inhibitor to a patient in need.

10. The method of claim 9, wherein the hematologic cancer is selected from myelodysplastic syndrome (MDS) and acute myeloid leukemia (AML).

11. The method of claim 10, wherein the CDK4/6 inhibitor is selected from trilaciclib, palbociclib, ribociclib, and any combination thereof.

12. The method of claim 11, wherein the CDK4/6 inhibitor is trilaciclib.

13. The method of claim 12, wherein administering the therapeutic amount of the CDK4/6 inhibitor prevents or mitigates the clonal hematopoiesis.

14. The method of claim 13, wherein the clonal hematopoiesis is associated with an administration of a chemotherapy to the patient.

15. The method of claim 14, wherein the chemotherapy is selected from carboplatin, etoposide, gemcitabine, FOLFOXIRI/bevacizumab, and any combination thereof.

16. The method of claim 14, further comprising administering the therapeutic amount of the CDK4/6 inhibitor to the patient concurrently with the chemotherapy.

17. The method of claim 9, wherein administering the therapeutic amount of a CDK4/6 inhibitor further prevents or mitigates myeloid neoplasms associated with the clonal hematopoiesis.

18. The method of claim 9, wherein administering the therapeutic amount of a CDK4/6 inhibitor further prevents or mitigates a cardiovascular disease associated with clonal hematopoiesis.
